(19) United States
(12) Patent Application Publication
(10) Pub. No.: US 2022/0134008 A1
(43) Pub. Date: May 5, 2022

(54) APPLICATOR SYRINGE CONTAINING A STERILE SOLUTION HAVING A MEDICAL ACTIVE SUBSTANCE, AND METHOD FOR PREPARATION OF SAME

(71) Applicant: Klaus STEGEMANN, Hamburg (DE)

(72) Inventor: Klaus STEGEMANN, Hamburg (DE)

(21) Appl. No.: 17/298,085

(22) PCT Filed: Nov. 28, 2019
(86) PCT No.: PCT/EP2019/082944

§ 371 Date: Nov. 15, 2021

(30) Foreign Application Priority Data
Nov. 30, 2018 (EP) 18209583.6

(51) Int. Cl. A61M 5/31; A61M 5/315

(52) U.S. Cl. CPC A61M 5/3134; A61M 5/315; A61M 2205/0205; A61M 2005/3104; A61M 2202/0007; A61M 2210/1085

(57) ABSTRACT

An applicator syringe (1) is disclosed containing a sterile solution having a medical active substance, in particular for introducing the solution containing the active substance via a urinary tract and/or bladder catheter into the bladder of a patient. The applicator syringe (1) includes a cylindrical jacket (2), which is closed by a syringe cover (3), which is integrally formed with the jacket (2) and includes an outlet opening (5) and is formed from a cycloolefin copolymer (COC). The applicator syringe (1) furthermore comprises a plunger tip made of an isobutene-isoprene rubber, which is halogenated in particular. The sterile solution containing the medical active substance is arranged in a cavity (6) delimited by the cylindrical jacket (2) having syringe cover (3) and the plunger tip.

### BACKGROUND

### Technical Field

The present invention relates to an applicator syringe containing a sterile solution having a medical active substance, in particular for introducing the solution via a urinary tract and/or bladder catheter into the bladder of a patient. It furthermore relates to a method for preparing such an applicator syringe containing a sterile solution having a medical active substance.

### Background Information

Supplying one or more solutions containing medical active substance(s) filled in applicator syringes for medical treatments to physicians or also to patients for self-medication is known.

Thus, for example, parasympatholytic agents, such as oxybutynin hydrochloride, are used for the treatment of overactive bladder. In particular, such parasympatholytic agents, such as oxybutynin hydrochloride, are used for relaxing the bladder in patients who can no longer empty their bladder in a natural way and are dependent on emptying the bladder via a catheter introduced into the urethra. Such patients include, for example and in particular, patients having paraplegia.

Parasympatholytic agents, such as medications containing oxybutynin hydrochloride, are in particular administered orally. The problem in the case of this dosage form is a delayed effect due to the delayed absorption via the gastrointestinal tract and furthermore side effects to be observed in this dosage form. These side effects include dryness of mouth, constipation, vision problems, fatigue, and dizziness. In particularly severe cases, delirium caused by the parasympatholytic agent, for example oxybutynin hydrochloride, can also occur.

Further dosage forms have thus already been developed which do not provide oral administration, but a transdermal administration of the active substance, for example using transdermal adhesive bandages or gels. This type of administration of the active substance can reduce side effects, but still results in a delayed effect of the medication.

There have furthermore also already been studies and practical implementations, in which a medication containing a parasympatholytic agent, for example oxybutynin hydrochloride, is instilled in the form of a solution directly into the bladder, in particular after emptying of the bladder via a catheter through the catheter already used for emptying the bladder. This dosage form has the significant advantage that not only are the side effects significantly reduced, but above all the effect of the medication takes place very soon, essentially immediately, since it is administered directly in the region of the bladder and no transport via the blood circulation is required.

In the previous applications of this type, in which a solution containing a parasympatholytic agent, for example an oxybutynin hydrochloride, is administered in an applicator syringe, which the user can administer independently after the emptying of the bladder via the placed bladder catheter, simple applicator syringes were used, which were produced from polyethylene (PE) or polypropylene (PP) and have a plunger tip on the plunger rod made of a simple rubber material. Furthermore, these applicator syringes have been used with a Luer lock fitting surrounding an outlet opening, onto which a matching adapter cone was screwed for the connection to the catheter respectively used by the patient for emptying his bladder.

### SUMMARY

In complex studies, the inventor has established that these previously known solutions are accompanied by various disadvantages. In particular, the inventor had to establish that in the previously used applicator syringes, in particular if they are subjected to a steam pressure treatment for a final sterilization and are heated in this case to the temperatures typical there of typically at least 120° C. (for example 121° C.), the concentration of the active substance, for example oxybutynin hydrochloride, in the solution contained in the applicator syringe changes, that this concentration decreases. This decrease of the active substance concentration could be attributed by the inventor in further complex studies to the fact that the previously used material of the applicator syringes, on the one hand the syringe body itself, namely the PP and the PE, on the other hand the plug or plunger tip, namely the conventional rubber, absorbs active substance contained in the solution, for example oxybutynin hydrochloride, in the course of the steam pressure sterilization and also holds it bound there after the steam pressure treatment.

The inventor has made it the object to counter this problem and provide an applicator syringe containing a sterile solution having a medical active substance, in particular containing a parasympatholytic agent, in particular containing oxybutynin hydrochloride, which can be sterilized in a steam pressure method without changing the concentration of the medical active substance in the solution.

This object is achieved according to the invention by an applicator syringe containing a sterile solution having a medical active substance, in particular for introducing the medication via a urinary tract and/or bladder catheter into the bladder of a patient, and having an applicator syringe having a cylindrical jacket closed on one side by a syringe cover formed integrally with the jacket and including an outlet opening, made of a cycloolefin copolymer (COC) and a plunger tip made of an isobutene-isoprene rubber (also referred to as butyl rubber), which is halogenated in particular, in which the solution containing the medical active substance is arranged in a cavity delimited by the cylindrical jacket having syringe cover and the plunger tip.

This is because the inventor has found in complex experiments that the materials COC and isobutene-isoprene rubber, which is halogenated in particular, provided by him for the applicator syringe, more precisely its jacket having syringe cover and for the plunger tip, do not absorb the medical active substance, for example a parasympatholytic agent such as oxybutynin hydrochloride, even in such conditions and in particular temperatures as are used for a final steam pressure sterilization, in any case after the steam pressure sterilization, the active substance concentration in the solution containing the medical active substance contained in the applicator syringe thus treated is not changed.

Thus for the first time a finished product corresponding to the required specifications for the sterility due to the final steam pressure sterilization can be offered, which contains the sterile solution having the medical active substance in the field concentration and can thus be used with the desired effect by the caregiver or an affected patient in a self-treatment.

The sterile solution can in particular contain as the medical active substance a parasympatholytic agent, in particular oxybutynin hydrochloride.

In one particularly advantageous refinement of the applicator syringe according to the invention, the outlet opening is formed in a stepped cone formed integrally with the jacket and the syringe cover having at least two, preferably at least three cylindrical sections having different diameters which become smaller toward a tip of the stepped cone. This design permits, for example, coupling of the applicator syringe to differently designed catheters having different connection cones, for example those for emptying the bladder. The integral formation of the stepped cone with the jacket and the syringe cover furthermore results in a significant improvement over the previously known designs having adapter cones fixed via a Luer lock connection on the applicator syringe. This is because these, this has also been established by the inventor in studies, do not remain sufficiently sealed during a final steam pressure sterilization of the filled applicator syringe connected to the adapter cone, so that a change of the solution containing the medical active substance, which is arranged in the applicator syringe, can also be the consequence here. In order to be able to connect a large number of catheters, for example catheters for emptying the bladder, as flexibly as possible to the stepped cone formed integrally with the jacket and the syringe cover in the advantageous refinement according to the invention, the stepped cone can include a large variety of cylindrical sections having different diameters which become smaller toward the tip of the stepped cone, for example 10 to 15 such cylindrical sections, in particular, for example, 12.

The material of the plunger tip can in particular be bromobutyl rubber that can be autoclaved.

According to the present invention, the medical active substance contained in the sterile solution can in particular be a parasympatholytic agent, in particular oxybutynin hydrochloride. In particular this active substance can be selected for the application for relaxing the bladder and dosed accordingly in the sterile solution.

For a medication for a relaxation of an overactive bladder to be carried out using the sterile solution with which the applicator syringe is filled, in particular sterile solutions containing a parasympatholytic agent, in particular oxybutynin hydrochloride, having a proportion of the mentioned active substance of 0.01 to 1.0 wt. %, preferably of 0.05 to 0.5 wt. %, in particular of 0.08 to 0.12 wt. %, advantageously 0.1 wt. % have proven to be suitable.

The sterile solution which is arranged in the cavity of the applicator syringe can advantageously be a NaCl solution containing oxybutynin hydrochloride. This can in particular be an isotonic NaCl solution containing oxybutynin hydrochloride.

A suitable amount, a suitable volume, of the sterile solution containing a medical active substance for a single medication can in particular be 1 to 50 mL, preferably 5 to 20 mL, in particular 8 to 12 mL, particularly advantageously 10 mL. This also applies in particular for sterile solutions as designated above, containing a parasympatholytic agent, in particular oxybutynin hydrochloride, in a proportion as specified above.

The applicator syringe filled with the sterile solution containing the medical active substance is in particular closed in a delivery state at its outlet opening by a closure element, in particular a closure plug or a closure cap, wherein this closure element consists of an isobutene-isoprene rubber, which is halogenated in particular. It can be formed in particular from a bromobutyl rubber that can be autoclaved. The closure element prevents premature escape of the sterile solution containing the medical active substance from the cavity of the application syringe and ensures that the region around the outlet opening which is covered by the closure plug or the closure cap is protected from microbial contamination, and thus remains sterile after a sterilization process up to a packaging of the applicator syringe in, for example, a blister pack. The designated material selection for the closure plug or the closure cap in turn again has the result that the closure element does not absorb active substance, for example, parasympatholytic agent, for example oxybutynin hydrochloride, from the solution in a final steam pressure sterilization process and therefore the active substance concentration in the sterile solution is not changed.

The applicator syringe according to the invention filled with the solution containing the medical active substance is in particular finally sterilized in the process and subsequently packaged, for example in a blister pack, so that when the user removes the applicator syringe from the package immediately before use, the syringe content is then sterile and free of germs and germs are thus prevented from being entrained into the urethral and bladder tract, for example during a treatment of the bladder.

Furthermore, a method for providing an applicator syringe containing a sterile solution having a medical active substance is also specified according to the invention, in particular for introducing the sterile solution via a urinary tract and/or bladder catheter into the bladder of a patient, which applicator syringe is formed as described above. The method comprises the following steps:

A sterile applicator syringe including a cylindrical jacket, which is closed on one side by a syringe cover integrally formed with the material and including an outlet opening, made of a cycloolefin copolymer (COC) and including a plunger tip made of an isobutene-isoprene rubber, which is halogenated in particular, is provided. Furthermore, a solution containing the medical active substance, for example a parasympatholytic agent, such as oxybutynin hydrochloride, is provided. This solution containing the medical active substance can in particular be formed according to the above specifications made in the description of the applicator syringe, thus having a NaCl solution, which is isotonic in particular, and/or in the above-specified concentrations of a parasympatholytic agent, such as oxybutynin hydrochloride, as the active substance. A cavity of the applicator syringe delimited by the cylindrical jacket having syringe cover and by the plunger tip is filled using the solution containing the active substance. This can in particular be filling of the cavity using a volume of the solution as specified above in the description of the applicator syringe. The outlet opening of the applicator syringe is closed using a closure element made of an isobutene-isoprene rubber, which is halogenated in particular, for example a bromobutyl rubber that can be autoclaved. The unit thus formed, thus the applicator syringe filled using the solution containing the active substance enclosed at its outlet opening, is finally steam pressure sterilized in an autoclave.

Following the steam pressure sterilization, the then sterilized applicator syringe filled with solution containing active substance and closed using the closure element can be packaged for delivery, storage, and the transport to the patient and possibly a retention there.

In particular an applicator syringe as described above can be provided with a stepped cone formed integrally with the jacket and the syringe cover having at least two, preferably at least three cylindrical sections having different diameters which become smaller toward a tip of the stepped cone. The stepped cone can in particular in turn be designed in a way as described and explained above on the basis of the description of the applicator syringe.

The steam pressure sterilization in the autoclave can be carried out for at least 10 minutes, in particular at least 15 minutes, in particular for 20 minutes, and preferably takes place at at least 115° C., in particular at least 120° C., in particular at precisely 121° C.

To confirm the effect according to the invention, comparison experiments were carried out for the autoclaving of a, for example, syringe filled with an oxybutynin hydrochloride solution made of polypropylene having a polyisoprene plunger tip plug and a correspondingly filled syringe made of cycloolefin copolymer (COC) having a bromobutyl rubber plunger tip plug. The different plunger tip plugs were selected according to their respective matching shape or their diameter for the corresponding syringe body. Corresponding syringes were filled with a 0.1% oxybutynin hydrochloride solution and steam sterilized for 30 minutes at 121° C. in a pass-through autoclave. The results are shown in Tables 1 and 2.

In this case, following the autoclaving, not only was the oxybutynin content of the solutions studied and compared to the target content (0.1%), but it was also ascertained to what extent contaminants of the oxybutynin solution are produced by the autoclaving of the solution in the syringe.

TABLE 1
results of autoclaving a polypropylene syringe
filled with a 0.1% oxybutynin hydrochloride solution
having a polyisoprene plunger tip plug

| | Oxybutinin-HCl | Contaminant |
|---|---|---|
| | content [%] | D [%] |
| | Specifikation: 95-105% | Specifikation: ≤0.2% |
| Sample 1 | 88.7 | 0.660 |
| Sample 2 | 90.6 | 0.680 |
| Sample 3 | 90.4 | 0.710 |
| Sample 4 | 90.7 | 0.700 |
| Sample 5 | 90.3 | 0.720 |
| Mean value | 90.14 | 0.6940 |
| Standard deviation | 0.82 | 0.0241 |
| Variation coefficient | 0.91 | 3.47 |
| Lower confidence | 89.28 | 0.6687 |
| interval (P = 95%) | | |
| Upper confidence | 91.00 | 0.7193 |
| interval (P = 95%) | | |

TABLE 2
results of autoclaving a COC syringe filled with a 0.1% oxybutynin
hydrochloride solution having a bromobutyl rubber plunger tip plug

| | Oxybutinin-HCl | Contaminant |
|---|---|---|
| | content [%] | D [%] |
| | specification: 95-105% | specification: ≤0.2% |
| Sample 1 | 99.2 | 0.060 |
| Sample 2 | 98.9 | 0.070 |
| Sample 3 | 98.6 | 0.060 |
| Sample 4 | 99.7 | 0.070 |
| Sample 5 | 98.6 | 0.070 |
| Mean value | 99.00 | 0.0660 |
| Standard deviation | 0.46 | 0.0055 |
| Variation coefficient | 0.47 | 8.30 |
| Lower confidence | 98.51 | 0.0603 |
| interval (P = 95%) | | |
| Upper confidence | 99.49 | 0.0717 |
| interval (P = 95%) | | |

Specifications which are to apply for the finished product are listed with “specification” in Tables 1 and 2.

It is apparent that the polypropylene syringe is not suitable to be autoclaved in the filled state, since a significant proportion of the active substance, in particular the oxybutynin, is absorbed by the syringe and/or degraded during the autoclaving. The study showed that only approximately 90% of the original active substance concentration, in particular oxybutynin concentration, is retained after the autoclaving, which thus significantly falls below the specified minimum proportion of 95%. Moreover, it is particularly noticeable that a high concentration of contaminant could be found in the solution autoclaved in this syringe, at approximately 0.7%, which significantly exceeds the specification of at most 0.2%. The studied polypropylene syringe is accordingly in no way suitable to be autoclaved with filled active substance solution.

In contrast, the experiments confirm the suitability of COC syringes for autoclaving with filled active substance, in particular oxybutynin. The specifications are clearly achieved at approximately 99% of the original active substance concentration, thus almost no absorption and/or no degradation of the active substance by the syringe, and only approximately 0.07% contaminants.

Because of the different plunger tip materials, these were additionally tested separately without the remaining syringe material to qualitatively assess an influence of the plunger tip material. For this purpose, respective plunger tip plugs of both materials were placed in a 0.1% oxybutynin hydrochloride solution and these were then steam sterilized for 30 minutes at 121° C. in the pass-through autoclave, therefore at the same conditions as the entire syringe. It is to be noted that the results of the studies on the plunger tip materials only provide qualitative information about the influence of the plunger tip material, but quantitative information cannot be obtained, since the contact surface between active substance solution and plunger tip material in the case of a plunger tip plug placed in the active substance solution is increased multiple times over that between active substance solution and plunger tip plug inserted into a syringe. The results are shown in Table 3.

TABLE 3
comparison of the results of the individual
syringes and the head materials

| | | Mean value of | |
|---|---|---|---|
| | | oxybutynin | Mean value of |
| | | concentration | contaminant D |
| Syringe | Syringe components | [%] | [%] |
| Polypropylene | Syringe with plunger | 90.14 | 0.69 |
| syringe | tip | | |
| | Polyisoprene | 92.74 | 0.08 |
| | plunger tip plug | | |
| COC syringe | Syringe with plunger | 99.0 | 0.07 |
| | tip | | |
| | Bromobutyl plunger | 97.8 | 0.06 |
| | tip plug | | |

It is apparent that the bromobuty plunger tip plug also has improved properties over the polyisoprene plunger tip plug, but these result as less in comparison to the improvement between the two tested syringes. If one observes the polypropylene syringe having the polyisoprene plunger tip plug, it is thus established that the obtained active substance proportion is less in the experiment carried out using the complete syringe than that which is obtained in the experiment carried out with separate plunger tip plug. In particular, a significant difference results in the concentration of the contaminants. Absorption and/or degradation of the active substance are therefore essentially to be attributed to the syringe material of the polypropylene syringe.

An inverted picture results for the COC syringe, the separate plunger tip plug even shows worse behavior than the complete syringe here, so that once again a suitability of COC as the syringe material for syringes filled with active substance and subsequently autoclaved results even more clearly here. In particular, the influence of the plunger tip materials has to be assessed as minor due to the small contact area proportion within the syringe.

This is also shown particularly from the measured content of contaminants. Only the polypropylene syringe shows an elevated value here, therefore a harmful interaction between active substance and syringe surface, for example due to a degradation of the active substance.

The results therefore clearly show the advantages of COC as a syringe material, in particular independently of the plunger tip material, for syringes filled with active substance and subsequently autoclaved.

### BRIEF DESCRIPTION OF THE SEVERAL VIEWS OF THE DRAWINGS

Further advantages and features of the invention result from the following description of an exemplary embodiment on the basis of the appended figures. In the figures:

FIG. 1 shows a three-dimensional view of an applicator syringe according to the invention (without the plunger rod with plunger tip);

FIG. 2 shows a sectional illustration of the applicator syringe shown in FIG. 1 (again without the plunger rod with plunger tip); and

FIG. 3 shows an enlarged view of the region identified by III in FIG. 2.

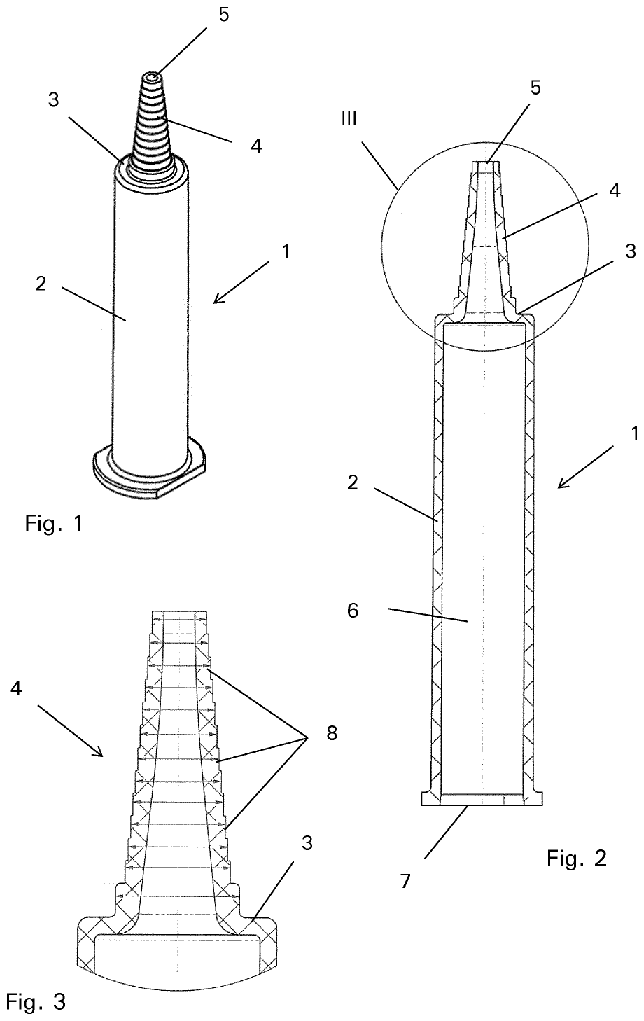

### DETAILED DESCRIPTION

A possible exemplary embodiment of an applicator syringe according to the invention is shown in schematic illustrations in the figures. The figures are not to scale and also do not include all elements illustrated required for the formation of an applicator syringe. In particular, a plunger rod having the plunger tip arranged thereon is not shown in the figures, as is part of the applicator syringe. Such a plunger rod is sufficiently known in its shaping to a person skilled in the art, however, so that reference can be made to the known designs. The special feature of the applicator syringe according to the invention is also not the shaping, but rather the material of the plunger tip.

An applicator syringe (shown as mentioned without the plunger rod with plunger tip) is generally identified in the figures with the reference sign 1. This applicator syringe 1 includes a jacket 2, with which a syringe cover 3 is integrally formed. Furthermore, a stepped cone 4, which is composed from a plurality of cylindrical sections 8 placed on one another having diameters each becoming smaller starting from the syringe cover 3 up to a tip of the stepped cone 4, is integrally formed on the syringe cover 3 in the exemplary embodiment shown. In the exemplary embodiment shown, these are a total of twelve such sections 8, which merge integrally via a neck into the syringe cover 3.

This stepped cone 4 integrally formed on the syringe cover 3 and thus connected to the material 2 permits connecting of the applicator syringe 1 to conical connection openings of catheters, for example those for emptying bladders, having very different shapes.

The special feature of the applicator syringe 1 according to the invention is that it is manufactured, for example injection molded, on the one hand, integrally with the jacket 2, the syringe cover 3, and the stepped cone 4 and from a special material, namely a cycloolefin copolymer (COC), and includes a plunger tip (not shown in the figures, inserted in a typical way into the jacket 2 from a rear end 7) made of a special rubber, namely an isobutene-isoprene rubber, which is halogenated in particular, for example a bromobutyl rubber that can be autoclaved. Furthermore, it is a special feature that this applicator syringe 1 contains, in a cavity 6 formed in the jacket 2 and delimited by the jacket 2, the syringe cover 3, and the plunger tip (not shown in greater detail), a sterile solution containing a medical active substance, in particular a solution containing a parasympatholytic agent, such as oxybutynin hydrochloride in particular, in particular an NaCl solution containing oxybutynin hydrochloride, in particular an isotonic NaCl solution containing oxybutynin hydrochloride is filled. In the course of the preparation, the applicator syringe 1 according to the invention, the outlet opening 5 of which has been closed in particular beforehand by a cap, which is placed on the stepped cone 4 and consists of an isobutene-isoprene rubber, which is halogenated in particular, preferably of bromobutyl rubber that can be autoclaved, has been sterilized in particular in a final step, in particular in a steam pressure sterilization in an autoclave, for example for 20 minutes at 121° C. The special material selection for the material of the jacket 2 with integrally formed syringe cover 3 and stepped cone 4 and also for the material of the plunger tip (and of the optionally usable closure in the form of a closure cap) enables no change of the active substance contained in the cavity 6, in particular with respect to its concentration in the sterile solution, to take place during this final sterilization process. The selected materials do not absorb this active substance, for example a parasympatholytic agent, for example oxybutynin hydrochloride, from the solution even at the temperatures set during the steam pressure sterilization and do not absorb it, so that no change occurs of the active substance concentration of the active substance in the solution, which is to be observed in known applicator syringe is from the prior art.

The concentration of a sterile solution containing a parasympatholytic agent, for example oxybutynin hydrochloride, in the cavity 6 can in particular be between 0.01 wt. % and 1.0 wt. %. In the exemplary embodiment described here, it is preferably 0.1 wt. %. The volume of the sterile solution received in the cavity 6 can be in particular between 1 mL and 50 mL and is preferably 10 mL in the exemplary embodiment shown.

The advantage that the stepped cone 4 is integrally formed with the syringe cover 3 is that at this point a leak to be observed in known applicator syringe is from the prior art cannot occur, so that influencing of the solution located in the cavity 6, in particular of the medical active substance dissolved therein, for example in the form of a parasympatholytic agent, such as oxybutynin hydrochloride, also cannot take place here in the final steam pressure sterilization step.

It is also clear once again from the above description of the exemplary embodiment which special advantages accompany the design according to the invention, in that this in particular enables a final sterilization of the applicator syringe 1 filled with the solution containing the medical active substance in a steam pressure sterilization step in an autoclave, without this resulting in an impairment of the solution filled in the applicator syringe 1 and the active substance dissolved therein.

### LIST OF REFERENCE SIGNS

applicator syringe
jacket
syringe cover
stepped cone
outlet opening
cavity
rear end
cylindrical section

1. An applicator syringe containing a sterile solution having a medical active substance for introducing the sterile solution containing the medical active substance via a urinary tract or bladder catheter into a bladder of a patient, wherein the applicator syringe comprises:
a cylindrical jacket formed from a cycloolefin copolymer (COC);
a cavity defined by the cylindrical jacket;
a syringe cover integrally formed with the jacket, wherein one side of the cylindrical jacket is closed by the syringe cover;
an outlet opening defined by the syringe cover, said outlet opening being in fluid communication with the cavity; and
a plunger made of an isobutene-isoprene rubber, said plunger being movably received within the cavity;
wherein the sterile solution containing the medical active substance is received in the cavity delimited by the cylindrical jacket having syringe cover and the plunger.

2. The applicator syringe as claimed in claim 1, wherein a parasympatholytic agent is contained in the sterile solution as the medical active substance.

3. The applicator syringe as claimed in claim 2, wherein the parasympatholytic agent is contained in the sterile solution at a proportion of 0.01 to 1.0 wt. %.

4. The applicator syringe as claimed claim 1, wherein the sterile solution containing the medical active substance is an NaCl solution containing oxybutynin hydrochloride.

5. The applicator syringe as claimed in claim 4, wherein the sterile solution containing the medical active substance is an isotonic NaCl solution containing oxybutynin hydrochloride.

6. The applicator syringe as claimed in claim 1, wherein the outlet opening is formed in a stepped cone, which is integrally formed with the jacket and the syringe cover and the stepped cone has at least two cylindrical sections having different diameters which become smaller toward a tip of the stepped cone.

7. The applicator syringe as claimed in claim 6, wherein the stepped cone includes ten to fifteen cylindrical sections having different diameters which become smaller toward the tip of the stepped cone.

8. The applicator syringe as claimed in claim 1, wherein the plunger tip is made of a bromobutyl rubber that is able to be autoclaved.

9. The applicator syringe as claimed in claim 1, wherein the sterile solution contains oxybutynin hydrochloride as the parasympatholytic agent, and the sterile solution is of a volume of 1 to 50 mL in the cavity.

10. The applicator syringe as claimed in claim 1, wherein the outlet opening is closed using a closure element made of an isobutene-isoprene rubber.

11. The applicator syringe as claimed in claim 1, wherein the applicator syringe is packaged in a sterilized manner.

12. A method for providing an applicator syringe containing a medication for the introduction of the medication via a urinary tract or bladder catheter into the bladder of the patient, as claimed in claim 1, wherein the method has the following steps:
providing the sterile applicator syringe, which includes the cylindrical jacket that is closed on the one side by the syringe cover, which is integrally formed with the jacket and includes the outlet opening, and the applicator syringe is formed from the cycloolefin copolymer (COC), and the plunger made of isobutene-isoprene rubber;
providing a solution containing oxybutynin hydrochloride as a medication to form the sterile solution containing the medical active substance;
filling the cavity of the applicator syringe delimited by the cylindrical jacket having the syringe cover and the plunger using the solution containing oxybutynin hydrochloride;
closing the outlet opening of the applicator syringe using the closure element made of isobutene-isoprene rubber; and
steam pressure sterilizing the applicator syringe filled with the solution containing oxybutynin hydrochloride and closed using the closure element in an autoclave.

13. The method as claimed in claim 12, wherein the step of providing the sterile applicator syringe further comprises integrally forming a stepped cone with the jacket and the syringe cover and providing at least two cylindrical sections in the stepped cone, wherein the at least two cylindrical sections have different diameters which become smaller toward a tip of the stepped cone.

14. The method as claimed in claim 12, wherein the steam pressure sterilizing is carried out in the autoclave for at least 10 minutes, at at least 115° C.

15. The method as claimed in claim 12, further comprising the step of packaging the applicator syringe, which is steam pressure sterilized, is filled with the solution containing oxybutynin hydrochloride, and is closed using the closure element.

16. The applicator syringe as claimed in claim 1, wherein the plunger is made of a halogenated isobutene-isoprene rubber.

17. The applicator syringe as claimed in claim 2, wherein the parasympatholytic agent is oxybutynin hydrochloride.

18. The applicator syringe as claimed in claim 3, wherein the parasympatholytic agent is contained in the sterile solution at a proportion of 0.05 to 0.5 wt. %.

19. The applicator syringe as claimed in claim 6, wherein the stepped cone has at least three cylindrical sections having different diameters which become smaller toward the tip of the stepped cone.

20. The applicator syringe as claimed in claim 9, wherein the sterile solution contains oxybutynin hydrochloride as the parasympatholytic agent, and the sterile solution is of a volume of 5 to 25 mL in the cavity.
